(19) United States
(12) Patent Application Publication
(10) Pub. No.: US 2010/0317690 A1
(43) Pub. Date: Dec. 16, 2010

(54) TREATMENT OF PROTEIN FOLDING DISORDERS

(75) Inventors: Akane Kawamura, Abingdon (GB); Alan Geoffrey Roach, Abingdon (GB); Francis Xavier Wilson, Abingdon (GB); Jonathon Mark Tinsley, Abingdon (GB); Robert Nash, Abingdon (GB); Richard Storer, Abingdon (GB)

Correspondence Address: HESLIN ROTHENBERG FARLEY & MESITI PC, 5 COLUMBIA CIRCLE, ALBANY, NY, 12203, US

(73) Assignee: SUMMIT CORPORATION PLC

(21) Appl. No.: 12/744,029

(22) PCT Filed: Nov. 20, 2008
(86) PCT No.: PCT/GB08/03885

§ 371 Date: Aug. 27, 2010

(30) Foreign Application Priority Data
Nov. 21, 2007 (GB) 0722792.9
Nov. 21, 2007 (GB) 0722793.7
Nov. 21, 2007 (GB) 0722794.5

(51) Int. Cl. A61K 31/46; A61K 31/403; A61K 31/4015; A61K 31/435; A61K 31/445; C07D 487/02; C07D 207/12; C07D 207/24; C07D 451/06; C07D 455/02; C07D 211/40; A61P 43/00; A61P 3/00

(52) U.S. Cl. 514/304; 514/413; 514/425; 514/299; 514/315; 514/306; 548/453; 548/542; 546/131; 546/138; 546/183; 546/243

(57) ABSTRACT

Described are various compounds and methods for the treatment of disorders arising from aberrant protein folding, including in particular lysosomal storage diseases. In particular, polyhydroxylated alkaloids and imino sugars which are pharmacoperones of an enzyme and which do not bind to a catalytic site of said enzyme are described.

### FIELD OF THE INVENTION

This invention relates to compounds and methods for the treatment of various disorders arising from aberrant protein folding, including in particular lysosomal storage diseases.

### BACKGROUND OF THE INVENTION

### Protein Folding Disorders and Lysosomal Storage Disease

Abnormalities in protein folding lead to many different diseases (see e.g. Welch and Brown (1996) Cell Stress and Chaperones 1(2): 109-115, Table 1 and Thomas et al. (1995) TIBS 20: 456-459 for a review). Genetically inherited diseases (including various lysosomal storage disorders) often arise from point mutations or deletions which produce aberrantly folding gene products which are partially active, not targeted to the appropriate subcellular compartment(s), unsecreted or rapidly degraded. In many such hereditary disorders, the cellular quality control system retains (and often destroys or recycles) the mutant proteins in the endoplasmic reticulum. This process may give rise to a pathological protein deficiency even in cases where the function of the protein is only partially impaired.

Lysosomal storage disorders are a group of diseases which arise from abnormal metabolism of various substrates, including glycosphingolipids, glycogen, mucopolysaccharides and glycoproteins. The metabolism of such compounds normally occurs in the lysosome and the process is regulated in a stepwise process by various degradative enzymes. Therefore, a deficient activity in any one enzyme can impair the entire process and result in the accumulation of particular substrates. Listed below are a number of lysosomal storage disorders and the corresponding defective enzymes:

| | |
|---|---|
| Pompe's disease: | Acid alpha-glucosidase |
| Gaucher's disease: | Acid beta-glucosidase or |
| | glucocerebrosidase |
| Fabry's disease: | alpha-Galactosidase A |
| GMI-gangliosidosis: | Acid beta-galactosidase |
| Tay-Sachs' disease: | beta-Hexosaminidase A |
| Sandhoff's disease: | beta-Hexosaminidase B |
| Niemann-Pick's disease: | Acid sphingomyelinase |
| Krabbe's disease: | Galactocerebrosidase |
| Farber's disease: | Acid ceramidase |
| Metachromatic leukodystrophy: | Arylsulfatase A |
| Hurler-Scheie's disease: | alpha-L-Iduronidase |
| Hunter's disease: | Iduronate-2-sulfatase |
| Sanfilippo's disease A: | Heparan N-sulfatase |
| Sanfilippo's disease B: | alpha-N-Acetylglucosaminidase |
| Sanfilippo's disease C: | Acetyl-CoA: alpha-glucosaminide |
| | N-acetyltransferase |
| Sanfilippo's disease D: | N-Acetylglucosamine-6-sulfate |
| | sulfatase |
| Morquio's disease A: | N-Acetylgalactosamine-6-sulfate |
| | sulfatase |
| Morquio's disease B: | Acid beta-galactosidase |
| Maroteaux-Lamy's disease: | Arylsulfatase B |
| Sly's disease: | beta-Glucuronidase |
| alpha-Mannosidosis: | Acid alpha-mannosidase |
| beta-Mannosidosis: | Acid beta-mannosidase |
| Fucosidosis: | Acid alpha-L-fucosidase |
| Sialidosis: | Sialidase |
| Schindler-Kanzaki's disease: | alpha-N-acetylgalactosaminidase |

### Pharmacoperones and ASSC Therapy of Lysosomal Storage Disorders

It has recently been discovered that certain small molecules can serve as a molecular scaffolding and cause otherwise-misfolded mutant proteins to fold and route correctly within the cell. Such molecules have been dubbed “chemical chaperones”, “pharmaceutical chaperones” or “pharmacoperones”. In particular, it has been recognised that competitive inhibitors of the mutant enzymes implicated in various lysosomal storage disorders can, at subinhibitory concentrations, act as “Active-Site-Specific Chaperones” or ASSCs by either inducing or stabilizing the proper conformation of the mutant enzyme by specific binding to the catalytic site (see Fan (2007) Iminosugars as active-site-specific chaperones for the treatment of lysosomal storage disorders, In Iminosugars From Synthesis to Therapeutic Applications: Compain, Philippe/Martin, Olivier R. (eds.) ISBN-13: 978-0-470-03391-3—John Wiley & Sons).

Various imino sugars have been identified as ASSCs and their specific binding to the catalytic active site of an enzyme implicated in lysosomal storage diseases exploited to form the basis of a new form of therapy dubbed active-site-specific chaperone therapy (see e.g. U.S. Pat. No. 6,583,158, U.S. Pat. No. 6,589,964 and U.S. Pat. No. 6,599,919). ASSC therapy uses low concentrations of potent enzyme inhibitors to enhance the folding and activity of mutant proteins in specific LSDs. This approach was first tested in Fabry's disease, where 1-deoxy-galactononjirimycin (DGJ), an inhibitor of alpha-galactosidase A, was used to enhance the residual alpha-galactosidase activity in cell lines from Fabry's disease patients (see U.S. Pat. No. 6,274,597 and U.S. Pat. No. 6,583,158). The ASSC strategy has been extended to other lysosomal storage diseases, including Gaucher's disease and GMI-gangliosidosis.

ASSC therapy is now currently under development for several LSDs, including Gaucher's disease, and offers several advantages over ERT or substrate deprivation therapy. Most notably, since the active site inhibitors used in ASSC are specific for the disease-causing enzyme, the therapy is targeted to a single protein and metabolic pathway, unlike substrate deprivation therapy that inhibits an entire synthetic pathway. Like substrate deprivation therapy, the small molecule inhibitors for ASSC have the potential of crossing the blood brain barrier and could be used to treat neurological LSD forms. Moreover, in addition to enhancing the activity of the deficient enzymes associated with the LSDs, the ASSCs have also been demonstrated to enhance the activity of the corresponding wild-type enzyme (see U.S. Pat. No. 6,589,964) and so can be used adjunctively with enzyme replacement therapy in LSD patients.

However, ASSC therapy is complicated by the fact that therapeutic potential depends on a favourable ratio of inhibitory activity to chaperone activity: if the concentration of inhibitor required to promote proper folding approaches the inhibitory concentration then therapeutic utility is severely compromised. There have been some attempts to improve the chaperone:inhibitor ratio of various imino sugars by chemical means (see e.g. WO2004/037373), but such approaches are not generally applicable and have limited utility.

The present inventors have now discovered that certain polyhydroxylated alkaloids (including various imino sugars) can act as pharmacoperones in a catalytic site-independent manner. Thus, the problems associated with chaperone:inhibitor ratios are removed and a new class of pharmacoperones with an improved therapeutic index is provided.

### SUMMARY OF THE INVENTION

### First Aspect

According to a first aspect of the present invention there is provided a polyhydroxylated alkaloid which is a pharmacoperone of an enzyme and which does not bind to a catalytic site of said enzyme.

Thus, the pharmacoperone of the invention need not be a competitive inhibitor of said enzyme, so removing the problems associated with chaperone:inhibitor ratios associated with known pharmacoperones.

In preferred embodiments, the pharmacoperone is an activator of said enzyme. In such embodiments, the pharmacoperone may specifically bind an activating allosteric site on the enzyme.

In other embodiments, the pharmacoperone may be a non-competitive inhibitor of said enzyme. In such embodiments, the chaperone:inhibitor ratio may be favourable in view of the availability of the catalytic site. In such embodiments the pharmacoperone may specifically bind an inhibiting allosteric site on the enzyme.

In other embodiments, the pharmacoperone of the invention does not bind to the enzyme at all, but acts as an indirect chaperone via a chaperone effect attendant on binding to a protein (e.g. enzyme) which itself acts as a chaperone or co-chaperone of the enzyme.

Also contemplated is a pharmaceutical composition comprising the pharmacoperone of the invention together with a pharmaceutical excipient.

In another aspect, the invention contemplates the pharmacoperone of the invention for use in therapy or prophylaxis, for example for use in treating or preventing a disease or disorder arising from abnormal protein folding (e.g. a lysosomal storage disease).

In another aspect, the invention contemplates the use (for example for the manufacture of a medicament) of a polyhydroxylated alkaloid which is a pharmacoperone of a protein and which does not bind to a catalytic site of an enzyme (e.g. a pharmacoperone as defined above) for use in treating or preventing a disease or disorder arising from abnormal protein folding.

In another aspect, the invention contemplates a method of treating or preventing a disease or disorder arising from abnormal protein folding in a mammalian cell, said method comprising administering a polyhydroxylated alkaloid which is a pharmacoperone of a protein and which does not bind to a catalytic site of an enzyme (e.g. a pharmacoperone as defined above) in an amount effective to enhance normal folding of the protein.

The disease or disorder arising from abnormal protein folding may be a lysosomal storage disease, for example a lysosomal storage disease selected from: (a) Pompe's disease; (b) Gaucher's disease; (c) Fabry's disease; (d) GMI-gangliosidosis; (e) Tay-Sachs' disease; (f) Sandhoff's disease; (g) Niemann-Pick's disease; (h) Krabbe's disease; (i) Farber's disease; (j) Metachromatic leukodystrophy; (k) Hurler-Scheie's disease; (l) Hunter's disease; (m) Sanfilippo's disease A, B, C or D; (n) Morquio's disease A or B; (o) Maroteaux-Lamy's disease; (p) Sly's disease; (q) alpha-Mannosidosis; (r) beta-Mannosidosis; (s) Fucosidosis; (t) Sialidosis; and (u) Schindler-Kanzaki's disease.

The polyhydroxylated alkaloid is preferably a pharmacoperone of an enzyme selected from: (a) Acid alpha-glucosidase; (b) Acid beta-glucosidase; (c) glucocerebrosidase; (d) alpha-Galactosidase A; (e) Acid beta-galactosidase; (f) beta-Hexosaminidase A; (g) beta-Hexosaminidase B; (h) Acid sphingomyelinase; (i) Galactocerebrosidase; (j) Acid ceramidase; (k) Arylsulfatase A; (l) alpha-L-Iduronidase; (m) Iduronate-2-sulfatase; (n) Heparan N-sulfatase; (o) alpha-N-Acetylglucosaminidase; (p) Acetyl-CoA: alpha-glucosaminide N-acetyltransferase; (q) N-Acetylglucosamine-6-sulfate sulfatase; (r) N-Acetylgalactosamine-6-sulfate sulfatase; (s) Acid beta-galactosidase; (t) Arylsulfatase B; (u) beta-Glucuronidase; (v) Acid alpha-mannosidase; (w) Acid beta-mannosidase; (x) Acid alpha-L-fucosidase; (y) Sialidase; and (z) alpha-N-acetylgalactosaminidase.

Any polyhydroxylated alkaloid as herein defined finds application in the invention. Preferably, the polyhydroxylated alkaloid is a bicyclic polyhydroxylated alkaloid. In other aspects, the alkaloid may be selected from:

(a) a piperidine alkaloid;

(b) a pyrroline alkaloid;

(c) a pyrrolidine alkaloid;

(d) a pyrrolizidine alkaloid;

(e) an indolizidine alkaloid;

(f) a quinolizidine alkaloid;

(g) a nortropane alkaloid (e.g. a calystegine); and

(h) mixtures of any two or more of (a) to (g).

In preferred aspects, the alkaloid may be an imino sugar or imino sugar acid.

In yet another aspect, the alkaloid may be:

(a) a glycoside (e.g. glucoside) derivative;

(b) a branched alkyl derivative; or

(c) a derivative in which one or more of the hydroxyl group(s) are masked or protected.

The alkaloid preferably has a molecular weight of 100 to 400 Daltons. Most preferred are alkaloids having a molecular weight of 150 to 300 Daltons (e.g. 200 to 250 Daltons).

In another aspect the pharmacoperone may be a polyhydroxylated piperidine alkaloid that comprises the nucleus:

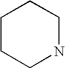

In another aspect the pharmacoperone may be a polyhydroxylated pyrrolidine alkaloid that comprises the nucleus:

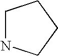

In another aspect the pharmacoperone may be a polyhydroxylated pyrrolizidine alkaloid that comprises the nucleus:

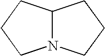

In another aspect the pharmacoperone may be a polyhydroxylated indolizidine alkaloid that comprises the nucleus:

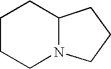

In another aspect the pharmacoperone may be a polyhydroxylated quinolizidine alkaloid that comprises the nucleus:

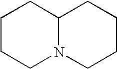

The invention also contemplates a process for producing polyhydroxylated alkaloid which is a pharmacoperone of an enzyme and which does not bind to a catalytic site of said enzyme comprising the steps of: (a) contacting said enzyme with a test substance; (b) detecting an increase of wild-type conformation of the enzyme in the presence of the test compound; and (c) detecting the absence of competitive inhibition by the test compound on said enzyme in the presence of substrate.

Also contemplated is a method of identifying a polyhydroxylated alkaloid useful for enhancing the in vivo activity of a mutant enzyme that folds aberrantly in vivo the activity of which is thereby deficient (e.g. an enzyme selected from enzymes (a) to (z) as listed above), which method comprises the steps of: (a) contacting said enzyme with a test substance; (b) detecting an increase of wild-type conformation of the enzyme in the presence of the test compound; and (c) detecting the absence of competitive inhibition by the test compound on said enzyme in the presence of substrate.

### Second Aspect

According to a second aspect of the present invention there is provided an imino sugar which is a pharmacoperone of an enzyme and which does not bind to a catalytic site of said enzyme.

Thus, the pharmacoperone of the invention need not be a competitive inhibitor of said enzyme, so removing the problems associated with chaperone:inhibitor ratios associated with known pharmacoperones.

In preferred embodiments, the pharmacoperone is an activator of said enzyme. In such embodiments, the pharmacoperone may specifically bind an activating allosteric site on the enzyme.

In other embodiments, the pharmacoperone may be a non-competitive inhibitor of said enzyme. In such embodiments, the chaperone:inhibitor ratio may be favourable in view of the availability of the catalytic site. In such embodiments the pharmacoperone may specifically bind an inhibiting allosteric site on the enzyme.

In other embodiments, the pharmacoperone of the invention does not bind to the enzyme at all, but acts as an indirect chaperone via a chaperone effect attendant on binding to a protein (e.g. enzyme) which itself acts as a chaperone or co-chaperone of the enzyme.

Also contemplated is a pharmaceutical composition comprising the pharmacoperone of the invention together with a pharmaceutical excipient.

In another aspect, the invention contemplates the pharmacoperone of the invention for use in therapy or prophylaxis, for example for use in treating or preventing a disease or disorder arising from abnormal protein folding (e.g. a lysosomal storage disease).

In another aspect, the invention contemplates the use (for example for the manufacture of a medicament) of an imino sugar which is a pharmacoperone of a protein and which does not bind to a catalytic site of an enzyme (e.g. a pharmacoperone as defined above) for use in treating or preventing a disease or disorder arising from abnormal protein folding.

In another aspect, the invention contemplates a method of treating or preventing a disease or disorder arising from abnormal protein folding in a mammalian cell, said method comprising administering an imino sugar which is a pharmacoperone of a protein and which does not bind to a catalytic site of an enzyme (e.g. a pharmacoperone as defined above) in an amount effective to enhance normal folding of the protein.

The disease or disorder arising from abnormal protein folding may be a lysosomal storage disease, for example a lysosomal storage disease selected from: (a) Pompe's disease; (b) Gaucher's disease; (c) Fabry's disease; (d) GMI-gangliosidosis; (e) Tay-Sachs' disease; (f) Sandhoff's disease; (g) Niemann-Pick's disease; (h) Krabbe's disease; (i) Farber's disease; (j) Metachromatic leukodystrophy; (k) Hurler-Scheie's disease; (l) Hunter's disease; (m) Sanfilippo's disease A, B, C or D; (n) Morquio's disease A or B; (o) Maroteaux-Lamy's disease; (p) Sly's disease; (q) alpha-Mannosidosis; (r) beta-Mannosidosis; (s) Fucosidosis; (t) Sialidosis; and (u) Schindler-Kanzaki's disease.

The imino sugar is preferably a pharmacoperone of an enzyme selected from: (a) Acid alpha-glucosidase; (b) Acid beta-glucosidase; (c) glucocerebrosidase; (d) alpha-Galactosidase A; (e) Acid beta-galactosidase; (f) beta-Hexosaminidase A; (g) beta-Hexosaminidase B; (h) Acid sphingomyelinase; (i) Galactocerebrosidase; (j) Acid ceramidase; (k) Arylsulfatase A; (l) alpha-L-Iduronidase; (m) Iduronate-2-sulfatase; (n) Heparan N-sulfatase; (o) alpha-N-Acetylglucosaminidase; (p) Acetyl-CoA: alpha-glucosaminide N-acetyltransferase; (q) N-Acetylglucosamine-6-sulfate sulfatase; (r) N-Acetylgalactosamine-6-sulfate sulfatase; (s) Acid beta-galactosidase; (t) Arylsulfatase B; (u) beta-Glucuronidase; (v) Acid alpha-mannosidase; (w) Acid beta-mannosidase; (x) Acid alpha-L-fucosidase; (y) Sialidase; and (z) alpha-N-acetylgalactosaminidase

Any imino sugar as herein defined finds application in the invention. Preferably, the imino sugar is a bicyclic polyhydroxylated alkaloid. In other aspects, the imino sugar is of a structural class selected from:

(i) a piperidine;

(j) a pyrroline;

(k) a pyrrolidine;

(l) a pyrrolizidine;

(m) an indolizidine;

(n) a quinolizidine;

(o) a nortropane alkaloid;

(p) mixtures of any two or more of (a) to (g).

In yet another aspect, the imino sugar may be:

(d) a glycoside (e.g. glucoside) derivative;

(e) an imino sugar acid;

(f) a branched alkyl derivative; or

(g) a derivative in which one or more of the hydroxyl group(s) are masked or protected.

The alkaloid preferably has a molecular weight of 100 to 400 Daltons. Most preferred are alkaloids having a molecular weight of 150 to 300 Daltons (e.g. 200 to 250 Daltons).

In another aspect the imino sugar may be a polyhydroxylated piperidine alkaloid that comprises the nucleus:

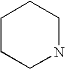

In another aspect the imino sugar may be a polyhydroxylated pyrrolidine alkaloid that comprises the nucleus:

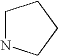

In another aspect the imino sugar may be a polyhydroxylated pyrrolizidine alkaloid that comprises the nucleus:

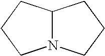

In another aspect the imino sugar may be a polyhydroxylated indolizidine alkaloid that comprises the nucleus:

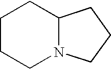

In another aspect the imino sugar may be a polyhydroxylated quinolizidine alkaloid that comprises the nucleus:

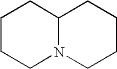

The invention also contemplates a process for producing an imino sugar which is a pharmacoperone of an enzyme and which does not bind to a catalytic site of said enzyme comprising the steps of: (a) contacting said enzyme with a test imino sugar; (b) detecting an increase of wild-type conformation of the enzyme in the presence of the test imino sugar; and (c) detecting the absence of competitive inhibition by the test imino sugar on said enzyme in the presence of substrate.

Also contemplated is a method of identifying an imino sugar useful for enhancing the in vivo activity of a mutant enzyme that folds aberrantly in vivo the activity of which is thereby deficient (e.g. an enzyme selected from enzymes (a) to (z) as listed above), which method comprises the steps of: (a) contacting said enzyme with a test imino sugar; (b) detecting an increase of wild-type conformation of the enzyme in the presence of the test imino sugar; and (c) detecting the absence of competitive inhibition by the test imino sugar on said enzyme in the presence of substrate.

### Third Aspect

According to a third aspect of the present invention there is provided a polyhydroxylated piperidine or pyrrolidine alkaloid which is a pharmacoperone of an enzyme and which does not bind to a catalytic site of said enzyme, the alkaloid comprising a nucleus selected from:

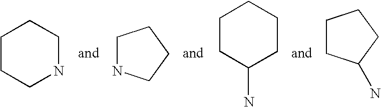

Thus, the pharmacoperone of the invention need not be a competitive inhibitor of said enzyme, so removing the problems associated with chaperone:inhibitor ratios associated with known pharmacoperones.

In preferred embodiments, the pharmacoperone is an activator of said enzyme. In such embodiments, the pharmacoperone may specifically bind an activating allosteric site on the enzyme. In other embodiments, the pharmacoperone may be a non-competitive inhibitor of said enzyme. In such embodiments, the chaperone:inhibitor ratio may be favourable in view of the availability of the catalytic site. In such embodiments the pharmacoperone may specifically bind an inhibiting allosteric site on the enzyme. In yet other embodiments, the pharmacoperone of the invention does not bind to the enzyme at all, but acts as an indirect chaperone via a chaperone effect attendant on binding to a protein (e.g. enzyme) which itself acts as a chaperone or co-chaperone of the enzyme.

Also contemplated is a pharmaceutical composition comprising the pharmacoperone of the invention together with a pharmaceutical excipient.

In another aspect, the invention contemplates the pharmacoperone of the invention for use in therapy or prophylaxis, for example for use in treating or preventing a disease or disorder arising from abnormal protein folding (e.g. a lysosomal storage disease).

In another aspect, the invention contemplates the use of a polyhydroxylated piperidine or pyrrolidine alkaloid which is a pharmacoperone of a protein and which does not bind to a catalytic site of an enzyme (e.g. a pharmacoperone as defined herein) for the manufacture of a medicament for use in treating or preventing a disease or disorder arising from abnormal protein folding.

In another aspect, the invention contemplates a method of treating or preventing a disease or disorder arising from abnormal protein folding in a mammalian cell, said method comprising administering a polyhydroxylated piperidine or pyrrolidine alkaloid which is a pharmacoperone of a protein and which does not bind to a catalytic site of an enzyme (e.g. a pharmacoperone as defined herein) in an amount effective to enhance normal folding of the protein.

The disease or disorder arising from abnormal protein folding may be a lysosomal storage disease, for example a lysosomal storage disease selected from: (a) Pompe's disease; (b) Gaucher's disease; (c) Fabry's disease; (d) GMI-gangliosidosis; (e) Tay-Sachs' disease; (f) Sandhoff's disease; (g) Niemann-Pick's disease; (h) Krabbe's disease; (i) Farber's disease; (j) Metachromatic leukodystrophy; (k) Hurler-Scheie's disease; (l) Hunter's disease; (m) Sanfilippo's disease A, B, C or D; (n) Morquio's disease A or B; (o) Maroteaux-Lamy's disease; (p) Sly's disease; (q) alpha-Mannosidosis; (r) beta-Mannosidosis; (s) Fucosidosis; (t) Sialidosis; and (u) Schindler-Kanzaki's disease.

The polyhydroxylated piperidine or pyrrolidine is preferably a pharmacoperone of an enzyme selected from: (a) Acid alpha-glucosidase; (b) Acid beta-glucosidase; (c) glucocerebrosidase; (d) alpha-Galactosidase A; (e) Acid beta-galactosidase; (f) beta-Hexosaminidase A; (g) beta-Hexosaminidase B; (h) Acid sphingomyelinase; (i) Galactocerebrosidase; (j) Acid ceramidase; (k) Arylsulfatase A; (l) alpha-L-Iduronidase; (m) Iduronate-2-sulfatase; (n) Heparan N-sulfatase; (o) alpha-N-Acetylglucosaminidase; (p) Acetyl-CoA: alpha-glucosaminide N-acetyltransferase; (q) N-Acetylglucosamine-6-sulfate sulfatase; (r) N-Acetylgalactosamine-6-sulfate sulfatase; (s) Acid beta-galactosidase; (t) Arylsulfatase B; (u) beta-Glucuronidase; (v) Acid alpha-mannosidase; (w) Acid beta-mannosidase; (x) Acid alpha-L-fucosidase; (y) Sialidase; and (z) alpha-N-acetylgalactosaminidase

In preferred aspects, the alkaloid may be an imino sugar or imino sugar acid. In yet another aspect, the alkaloid may be:

(h) a glycoside (e.g. glucoside) derivative;

(i) a branched alkyl derivative; or

(j) a derivative in which one or more of the hydroxyl group(s) are masked or protected.

The piperidine or pyrrolidine alkaloid preferably has a molecular weight of 100 to 400 Daltons. Most preferred are piperidine or pyrrolidine alkaloids having a molecular weight of 150 to 300 Daltons (e.g. 200 to 250 Daltons).

The invention also contemplates a process for producing polyhydroxylated piperidine or pyrrolidine alkaloid which is a pharmacoperone of an enzyme and which does not bind to a catalytic site of said enzyme comprising the steps of: (a) contacting said enzyme with a test substance; (b) detecting an increase of wild-type conformation of the enzyme in the presence of the test compound; and (c) detecting the absence of competitive inhibition by the test compound on said enzyme in the presence of substrate.

Also contemplated is a method of identifying a polyhydroxylated piperidine or pyrrolidine alkaloid useful for enhancing the in vivo activity of a mutant enzyme that folds aberrantly in vivo the activity of which is thereby deficient (e.g. an enzyme selected from enzymes (a) to (z) as listed above), which method comprises the steps of: (a) contacting said enzyme with a test substance; (b) detecting an increase of wild-type conformation of the enzyme in the presence of the test compound; and (c) detecting the absence of competitive inhibition by the test compound on said enzyme in the presence of substrate.

In all of the above aspects of the invention, and where the invention contemplates the use of a compound which is a pharmacoperone of a protein and which does not bind to a catalytic site of an enzyme, the compound (e.g. polyhydroxylated alkaloid or imino sugar) is a pharmacoperone of a protein and does not bind to a catalytic site of a lysosomal enzyme, for example an enzyme selected from: (a) Acid alpha-glucosidase; (b) Acid beta-glucosidase; (c) glucocerebrosidase; (d) alpha-Galactosidase A; (e) Acid beta-galactosidase; (f) beta-Hexosaminidase A; (g) beta-Hexosaminidase B; (h) Acid sphingomyelinase; (i) Galactocerebrosidase; (j) Acid ceramidase; (k) Arylsulfatase A; (l) alpha-L-Iduronidase; (m) Iduronate-2-sulfatase; (n) Heparan N-sulfatase; (o) alpha-N-Acetylglucosaminidase; (p) Acetyl-CoA: alpha-glucosaminide N-acetyltransferase; (q) N-Acetylglucosamine-6-sulfate sulfatase; (r) N-Acetylgalactosamine-6-sulfate sulfatase; (s) Acid beta-galactosidase; (t) Arylsulfatase B; (u) beta-Glucuronidase; (v) Acid alpha-mannosidase; (w) Acid beta-mannosidase; (x) Acid alpha-L-fucosidase; (y) Sialidase; and (z) alpha-N-acetylgalactosaminidase.

All of the above aspects also contemplate ex vivo processes for producing a polyhydroxylated alkaloid or an imino sugar which is a pharmacoperone of a mutant enzyme that folds aberrantly in vivo and which does not bind to a catalytic site of said mutant enzyme comprising the steps of: (a) contacting a cell extract comprising said mutant enzyme with a test polyhydroxylated alkaloid or test imino sugar; (b) detecting an increase of wild-type conformation of the enzyme in the presence of the test compound; and (c) determining whether said test polyhydroxylated alkaloid or test imino sugar binds to the active site of said mutant enzyme.

All of the above aspects also contemplate ex vivo methods of identifying a polyhydroxylated alkaloid or imino sugar useful for enhancing the in vivo activity of a mutant enzyme that folds aberrantly in vivo the activity of which is thereby deficient (e.g. an enzyme selected from enzymes (a) to (z) of claim 12), which method comprises the steps of: (a) contacting a cell extract comprising said mutant enzyme with a test polyhydroxylated alkaloid or test imino sugar; (b) detecting an increase of wild-type conformation of the enzyme in the presence of the test compound; and (c) determining whether said test polyhydroxylated alkaloid or test imino sugar binds to the active site of said mutant enzyme.

### DETAILED DESCRIPTION OF THE INVENTION

### Definitions and General Preferences

Where used herein and unless specifically indicated otherwise, the following terms are intended to have the following meanings in addition to any broader (or narrower) meanings the terms might enjoy in the art:

The term pharmacoperone is a term of art (from “pharmacological chaperone”) used to define a class of biologically active small molecules (sometimes also referred to in the art as “chemical chaperones”) that serve as molecular scaffolds, causing otherwise misfolded mutant proteins to fold and route correctly within the cell.

The terms derivative and pharmaceutically acceptable derivative as applied to the alkaloids of the invention define compounds which are obtained (or obtainable) by chemical derivatization of the parent alkaloids of the invention. The pharmaceutically acceptable derivatives are therefore suitable for administration to or use in contact with the tissues of humans without undue toxicity, irritation or allergic response (i.e. commensurate with a reasonable benefit/risk ratio). Preferred derivatives are those obtained (or obtainable) by alkylation, esterification or acylation of the parent alkaloids. The derivatives may act as pharmacoperones per se, or may be inactive until processed in vivo. In the latter case, the derivatives of the invention act as pro-drugs. Particularly preferred pro-drugs are ester derivatives which are esterified at one or more of the free hydroxyls and which are activated by hydrolysis in vivo. The pharmaceutically acceptable derivatives of the invention retain some or all of the chaperone activity of the parent compound. In some cases, the chaperone activity is increased by derivatization. Derivatization may also augment other biological activities of the alkaloid, for example bioavailability and/or glycosidase inhibitory activity and/or glycosidase inhibitory profile. For example, derivatization may increase glycosidase inhibitory potency and/or specificity and/or CNS penetration (e.g. penetration of the blood-brain barrier).

The term pharmaceutically acceptable salt as applied to the alkaloids of the invention defines any non-toxic organic or inorganic acid addition salt of the free base alkaloid which are suitable for use in contact with the tissues of humans and lower animals without undue toxicity, irritation, allergic response and which are commensurate with a reasonable benefit/risk ratio. Suitable pharmaceutically acceptable salts are well known in the art. Examples are the salts with inorganic acids (for example hydrochloric, hydrobromic, sulphuric and phosphoric acids), organic carboxylic acids (for example acetic, propionic, glycolic, lactic, pyruvic, malonic, succinic, fumaric, malic, tartaric, citric, ascorbic, maleic, hydroxymaleic, dihydroxymaleic, benzoic, phenylacetic, 4-aminobenzoic, 4-hydroxybenzoic, anthranilic, cinnamic, salicylic, 2-phenoxybenzoic, 2-acetoxybenzoic and mandelic acid) and organic sulfonic acids (for example methanesulfonic acid and p-toluenesulfonic acid). The drugs of the invention may also be converted into salts by reaction with an alkali metal halide, for example sodium chloride, sodium iodide or lithium iodide. Preferably, the alkaloids of the invention are converted into their salts by reaction with a stoichiometric amount of sodium chloride in the presence of a solvent such as acetone.

These salts and the free base compounds can exist in either a hydrated or a substantially anhydrous form. Crystalline forms of the alkaloids of the invention are also contemplated and in general the acid addition salts of the alkaloids are crystalline materials which are soluble in water and various hydrophilic organic solvents and which in comparison to their free base forms, demonstrate higher melting points and an increased solubility.

The term imino sugar defines a saccharide analogue in which the ring oxygen is replaced by a nitrogen.

In its broadest aspect, the present invention contemplates all optical isomers, racemic forms and diastereoisomers of the alkaloids described herein. Those skilled in the art will appreciate that, owing to the asymmetrically substituted carbon atoms present in the alkaloids of the invention, the alkaloids may be produced in optically active and racemic forms. Thus, references to the alkaloids of the present invention encompass the products as a mixture of diastereoisomers, as individual diastereoisomers, as a mixture of enantiomers as well as in the form of individual enantiomers.

### Alkaloids for Use According to the Invention

The compound of the invention may be an alkaloid as defined below.

The term alkaloid is used herein sensu stricto to define any basic, organic, nitrogenous compound which occurs naturally in an organism. In this sense, the term embraces naturally occurring imino sugars (see infra). However, it should be noted that the term alkaloid is also used herein sensu lato to define a broader grouping of compounds which include not only the naturally-occurring alkaloids, but also their synthetic and semi-synthetic analogues and derivatives. Thus, as used herein, the term alkaloid covers not only naturally-occurring basic, organic, nitrogenous compounds but also derivatives and analogues thereof which are not naturally occurring (and which may not be basic). In this context, the term imino sugar defines a saccharide (e.g. a mono- or disaccharide) analogue in which the ring oxygen is replaced by a nitrogen. As used herein, the term alkaloid also covers exocyclic amines in which the nitrogen is not present in the ring nucleus. Such exocyclic amines may be imino sugar analogues in which the ring nitrogen is absent and replaced with an exocyclic nitrogen. Such exocyclic amines may be piperidine or pyrrolidine alkaloid analogues in which the ring nitrogen is absent and replaced with an exocyclic nitrogen, so including piperidine analogues having the nucleus:

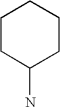

and pyrrolidine alkaloids having the nucleus:

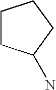

Most known alkaloids are phytochemicals, present as secondary metabolites in plant tissues (where they may play a role in defence), but some occur as secondary metabolites in the tissues of animals, microorganisms and fungi. There is growing evidence that the standard techniques for screening microbial cultures are inappropriate for detecting many classes of alkaloids (particularly highly polar alkaloids, see below) and that microbes (including bacteria and fungi, particularly the filamentous representatives) will prove to be an important source of alkaloids as screening techniques become more sophisticated.

Structurally, alkaloids exhibit great diversity. Many alkaloids are small molecules, with molecular weights below 250 Daltons. The skeletons may be derived from amino acids, though some are derived from other groups (such as steroids). Others can be considered as sugar analogues. It is becoming apparent (see Watson et al. (2001) Phytochemistry 56: 265-295) that the water soluble fractions of medicinal plants and microbial cultures contain many interesting novel polar alkaloids, including many carbohydrate analogues. Such analogues include a rapidly growing number of polyhydroxylated alkaloids.

Most alkaloids are classified structurally on the basis of the configuration of the N-heterocycle. Examples of some important alkaloids and their structures are set out in Kutchan (1995) The Plant Cell 7:1059-1070. Watson et al. (2001) Phytochemistry 56: 265-295 have classified a comprehensive range of polyhydroxylated alkaloids inter alia as piperidine, pyrroline, pyrrolidine, pyrrolizidine, indolizidine and nortropanes alkaloids (see FIGS. 1-7 of Watson et al. (2001), the disclosure of which is incorporated herein by reference).

Watson et al. (2001), ibidem also show that a functional classification of at least some alkaloids is possible on the basis of their glycosidase inhibitory profile: many polyhydroxylated alkaloids are potent and highly selective glycosidase inhibitors. These alkaloids can mimic the number, position and configuration of hydroxyl groups present in pyranosyl or furanosyl moieties and so bind to the active site of a cognate glycosidase, thereby inhibiting it. This area is reviewed in Legler (1990) Adv. Carbohydr. Chem. Biochem. 48: 319-384 and in Asano et al. (1995) J. Med. Chem. 38: 2349-2356.

As used herein, the term polyhydroxylated alkaloid defines a class of highly oxygenated alkaloids having at least 2,3, 4, 5, 6 or 7 (preferably 3, 4 or 5) free hydroxyl groups on the ring system nucleus.

As used herein, the term bicyclic polyhydroxylated alkaloid defines a class of highly oxygenated alkaloids having a double or fused ring nucleus (i.e. having two or more cyclic rings in which two or more atoms are common to two adjoining rings). Typically, such alkaloids have at least 3, 4, 5, 6 or 7 (preferably 3, 4 or 5) free hydroxyl groups on the ring system nucleus.

As used herein, the term polyhydroxylated piperidine alkaloid defines a highly oxygenated alkaloid (e.g. having at least 2 (preferably at least 3) free hydroxyl groups on the ring system nucleus) that comprises the nucleus:

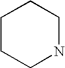

As used herein, the term polyhydroxylated pyrrolidine alkaloid defines a highly oxygenated alkaloid (e.g. having at least 2 (preferably at least 3) free hydroxyl groups on the ring system nucleus) that comprises the nucleus:

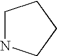

As used herein, the term polyhydroxylated pyrrolizidine alkaloid defines a highly oxygenated alkaloid (e.g. having at least 3, 4, 5, 6 or 7 (preferably 3, 4 or 5) free hydroxyl groups on the ring system nucleus) that comprises the nucleus:

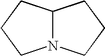

As used herein, the term polyhydroxylated indolizidine alkaloid defines a highly oxygenated alkaloid (e.g. having at least 3, 4, 5, 6 or 7 (preferably 3, 4 or 5) free hydroxyl groups on the ring system nucleus) that comprises the nucleus:

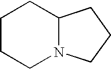

As used herein, the term polyhydroxylated quinolizidine alkaloid defines a highly oxygenated alkaloid (e.g. having at least 3, 4, 5, 6 or 7 (preferably 3, 4, 5 or 6) free hydroxyl groups on the ring system nucleus) that comprises the nucleus:

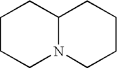

Yet other polyhydroxylated alkaloids for use according to the invention may comprise the nucleus:

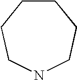

It has long been recognized that many alkaloids are pharmacologically active, and humans have been using alkaloids (typically in the form of plant extracts) as poisons, narcotics, stimulants and medicines for thousands of years. The therapeutic applications of polyhydroxylated alkaloids have been comprehensively reviewed in Watson et al. (2001), ibidem: applications include cancer therapy, immune stimulation, the treatment of diabetes, the treatment of infections (especially viral infections), therapy of glycosphingolipid lysosomal storage diseases and the treatment of autoimmune disorders (such as arthritis and sclerosis).

The alkaloid may be an imino sugar. Particularly preferred are polyhydroxylated imino sugar alkaloids. Preferred are imino sugars having a small molecular weight, since these may exhibit desirable pharmacokinetics. Thus, the imino sugar may have a molecular weight of 100 to 400 Daltons, preferably 150 to 300 Daltons and most preferably 200 to 250 Daltons. Also preferred are non-metabolizable imino sugars. Such sugars may exhibit extended tissue residence durations, and so exhibit favourable pharmacokinetics.

In a preferred embodiment, the imino sugar has the formula:

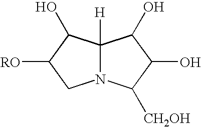

wherein R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof.

In another preferred embodiment the imino sugar has the formula:

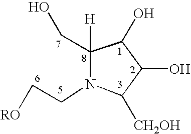

wherein R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof.

In such embodiments, most preferred are imino sugars having the formula:

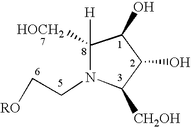

wherein R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof.

Examples of such preferred imino sugars include N-hydroxyethylDMDP having the formula:

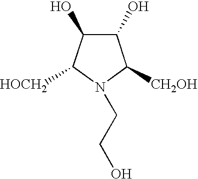

or a pharmaceutically acceptable salt or derivative thereof.

In another embodiment, the imino sugar has the formula:

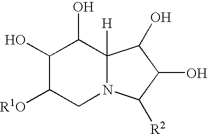

wherein R¹ is selected from the group comprising hydrogen, straight or branched, unsubstituted or substituted, saturated or unsaturated acyl, alkyl (e.g. cycloalkyl), alkenyl, alkynyl and aryl groups and R² is selected from hydrogen, hydroxy and alkoxy, or a pharmaceutically acceptable salt or derivative thereof.

In another embodiment, the imino sugar has the formula:

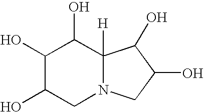

or a pharmaceutically acceptable salt or derivative thereof.

### Calystegines

A preferred class of polyhydroxylated alkaloid for use according to the invention are calystegines. These are polyhdroxylated nor-tropane alkaloids which have been reported to inhibit β-glucosidases, β-xylosidases and α-galactosidases (Asano et al., 1997, Glycobiology 7: 1085-1088). The calystegines are common in foods belonging to the Solanaceae that includes potatoes and aubergines (egg plant). The calystegines have been shown to inhibit mammalian glycosidases including human, rat and bovine liver enzymes. Attaching sugars to the calystegines such as in 3-0-β-D-glucopyranoside of 1α,2β,3α,6α-tetrahydroxy-nor-tropane (Calystegine B₁) (Griffiths, et al., 1996, Tetrahedron Letters 37: 3207-3208) can alter the glycosidase inhibition to include α-glucosidases and β-galactosidases.

Exemplary calystegines for use according to the invention include the compounds calystegine A₃, calystegine B₁ and calystegine B₂ shown below:

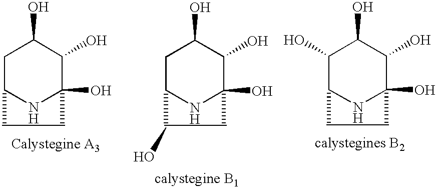

or pharmaceutically acceptable salts or derivatives (e.g. acyl derivatives) thereof.

Also suitable for use according to the invention are C-calystegines. These are pentahydroxycalystegines that possess the extra hydroxyl on the bridge as in calystegine B₁ and N-methylcalystegines have also been reported from plants including Lycium chinense (Watson et al., 2001, Phytochemistry 56, 265-295). Examples include compounds having the formulae shown below:

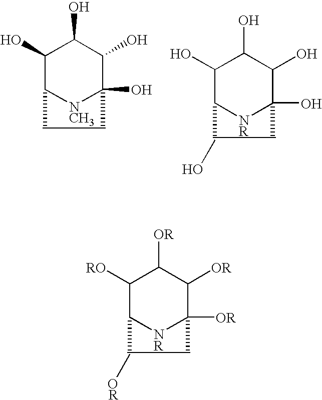

or pharmaceutically acceptable salts or derivatives (e.g. acyl derivatives) thereof.

### Imino Sugars for Use According to the Invention

The compound of the invention may be an imino sugar as defined below.

The term imino sugar defines a saccharide analogue in which the ring oxygen is replaced by a nitrogen.

As used herein, the term polyhydroxylated imino sugar defines a class of highly oxygenated imino sugars having at least 2, 3, 4, 5, 6 or 7 (preferably 3, 4 or 5) free hydroxyl groups on the ring system nucleus.

As used herein, the term bicyclic polyhydroxylated imino sugar defines a class of highly oxygenated imino sugars having a double or fused ring nucleus (i.e. having two or more cyclic rings in which two or more atoms are common to two adjoining rings). Typically, such imino sugars have at least 3, 4, 5, 6 or 7 (preferably 3, 4 or 5) free hydroxyl groups on the ring system nucleus.

As used herein, the term polyhydroxylated piperidine imino sugar defines a highly oxygenated imino sugar (e.g. having at least 2 (preferably at least 3) free hydroxyl groups on the ring system nucleus) that comprises the nucleus:

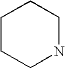

As used herein, the term polyhydroxylated pyrrolidine imino sugar defines a highly oxygenated imino sugar (e.g. having at least 2 (preferably at least 3) free hydroxyl groups on the ring system nucleus) that comprises the nucleus:

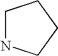

As used herein, the term polyhydroxylated pyrrolizidine imino sugar defines a highly oxygenated imino sugar (e.g. having at least 3, 4, 5, 6 or 7 (preferably 3, 4 or 5) free hydroxyl groups on the ring system nucleus) that comprises the nucleus:

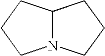

As used herein, the term polyhydroxylated indolizidine imino sugar defines a highly oxygenated imino sugar (e.g. having at least 3, 4, 5, 6 or 7 (preferably 3, 4 or 5) free hydroxyl groups on the ring system nucleus) that comprises the nucleus:

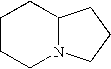

As used herein, the term polyhydroxylated quinolizidine imino sugar defines a highly oxygenated imino sugar (e.g. having at least 3, 4, 5, 6 or 7 (preferably 3, 4, 5 or 6) free hydroxyl groups on the ring system nucleus) that comprises the nucleus:

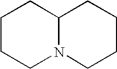

It has long been recognized that many imino sugars are pharmacologically active, and humans have been using imino sugars (typically in the form of plant extracts) as poisons, narcotics, stimulants and medicines for thousands of years. The therapeutic applications of polyhydroxylated imino sugars have been comprehensively reviewed in Watson et al. (2001), ibidem: applications include cancer therapy, immune stimulation, the treatment of diabetes, the treatment of infections (especially viral infections), therapy of glycosphingolipid lysosomal storage diseases and the treatment of autoimmune disorders (such as arthritis and sclerosis).

The imino sugar may be a polyhydroxylated alkaloid as herein defined. Preferred are imino sugars having a small molecular weight, since these may exhibit desirable pharmacokinetics. Thus, the imino sugar may have a molecular weight of 100 to 400 Daltons, preferably 150 to 300 Daltons and most preferably 200 to 250 Daltons. Also preferred are non-metabolizable imino sugars. Such sugars may exhibit extended tissue residence durations, and so exhibit favourable pharmacokinetics.

In a preferred embodiment, the imino sugar has the formula:

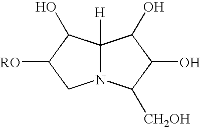

wherein R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof.

In another preferred embodiment the imino sugar has the formula:

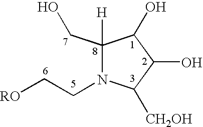

wherein R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof.

In such embodiments, most preferred are imino sugars having the formula:

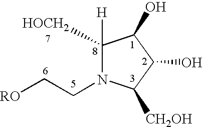

wherein R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof.

Examples of such preferred imino sugars include N-hydroxyethylDMDP having the formula:

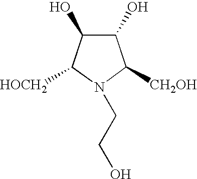

or a pharmaceutically acceptable salt or derivative thereof.

In another embodiment, the imino sugar has the formula:

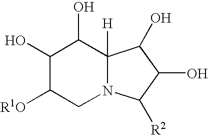

wherein R¹ is selected from the group comprising hydrogen, straight or branched, unsubstituted or substituted, saturated or unsaturated acyl, alkyl (e.g. cycloalkyl), alkenyl, alkynyl and aryl groups and R² is selected from hydrogen, hydroxy and alkoxy, or a pharmaceutically acceptable salt or derivative thereof.

In another embodiment, the imino sugar has the formula:

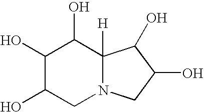

or a pharmaceutically acceptable salt or derivative thereof.

### Piperidine and Pyrrolidine Alkaloids for Use According to the Invention

The compound of the invention may be a piperidine or pyrrolidine alkaloid as defined below.

As used herein, the term polyhydroxylated piperidine or pyrrolidine alkaloid defines a class of highly oxygenated piperidine or pyrrolidine alkaloids having at least 2, 3, 4, 5, 6 or 7 (preferably 3, 4 or 5) free hydroxyl groups on the ring system nucleus.

As used herein, the term polyhydroxylated piperidine alkaloid defines a highly oxygenated alkaloid (e.g. having at least 2 (preferably at least 3) free hydroxyl groups on the ring system nucleus) that comprises the nucleus:

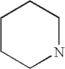

As used herein, the term polyhydroxylated pyrrolidine alkaloid defines a highly oxygenated alkaloid (e.g. having at least 2 (preferably at least 3) free hydroxyl groups on the ring system nucleus) that comprises the nucleus:

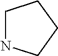

The piperidine or pyrrolidine alkaloid may be an imino sugar. Particularly preferred are polyhydroxylated imino sugar piperidine or pyrrolidine alkaloids. Preferred are imino sugars having a small molecular weight, since these may exhibit desirable pharmacokinetics. Thus, the imino sugar may have a molecular weight of 100 to 400 Daltons, preferably 150 to 300 Daltons and most preferably 200 to 250 Daltons. Also preferred are non-metabolizable imino sugars. Such sugars may exhibit extended tissue residence durations, and so exhibit favourable pharmacokinetics.

In another preferred embodiment the alkaloid has the formula:

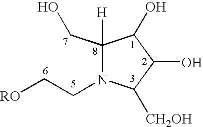

wherein R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. In such embodiments, most preferred are compounds having the formula:

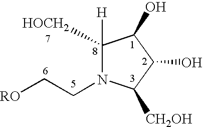

wherein R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof.

Examples of such preferred alkaloids include N-hydroxyethylDMDP having the formula:

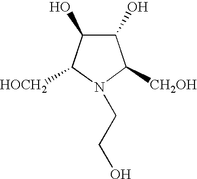

or a pharmaceutically acceptable salt or derivative thereof.

### Posology

The alkaloids of the present invention can be administered by oral or parenteral routes, including intravenous, intramuscular, intraperitoneal, subcutaneous, transdermal, airway (aerosol), rectal, vaginal and topical (including buccal and sublingual) administration.

The amount administered can vary widely according to the particular dosage unit employed, the period of treatment, the age and sex of the patient treated, the nature and extent of the disorder treated, and the particular compound selected.

Moreover, the alkaloids of the invention can be used in conjunction with other agents known to be useful in the treatment of diseases or disorders arising from protein folding abnormalities (as described infra) and in such embodiments the dose may be adjusted accordingly.

In general, the effective amount of the alkaloid administered will generally range from about 0.01 mg/kg to 500 mg/kg daily. A unit dosage may contain from 0.05 to 500 mg of the alkaloid, and can be taken one or more times per day. The alkaloid can be administered with a pharmaceutical carrier using conventional dosage unit forms either orally, parenterally, or topically, as described below.

The preferred route of administration is oral administration. In general a suitable dose will be in the range of 0.01 to 500 mg per kilogram body weight of the recipient per day, preferably in the range of 0.1 to 50 mg per kilogram body weight per day and most preferably in the range 1 to 5 mg per kilogram body weight per day.

The desired dose is preferably presented as a single dose for daily administration. However, two, three, four, five or six or more sub-doses administered at appropriate intervals throughout the day may also be employed. These sub-doses may be administered in unit dosage forms, for example, containing 0.001 to 100 mg, preferably 0.01 to 10 mg, and most preferably 0.5 to 1.0 mg of active ingredient per unit dosage form.

### Formulation

The alkaloid for use as pharmacoperone of the invention may take any form. It may be synthetic, purified or isolated from natural sources.

When isolated from a natural source, the pharmacoperone may be purified. In embodiments where the alkaloid is formulated together with a pharmaceutically acceptable excipient, any suitable excipient may be used, including for example inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc.

The pharmaceutical compositions may take any suitable form, and include for example tablets, elixirs, capsules, solutions, suspensions, powders, granules and aerosols.

The pharmaceutical composition may take the form of a kit of parts, which kit may comprise the composition of the invention together with instructions for use and/or a plurality of different components in unit dosage form.

Tablets for oral use may include the alkaloid of the invention, mixed with pharmaceutically acceptable excipients, such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatin capsules in which the pyrrolizidine compound of the invention is mixed with a solid diluent, and soft gelatin capsules wherein the active ingredient is mixed with water or an oil such as peanut oil, liquid paraffin or olive oil.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate.

The compounds of the invention may also be presented as liposome formulations.

For oral administration the pyrrolizidine compound of the invention can be formulated into solid or liquid preparations such as capsules, pills, tablets, troches, lozenges, melts, powders, granules, solutions, suspensions, dispersions or emulsions (which solutions, suspensions dispersions or emulsions may be aqueous or non-aqueous). The solid unit dosage forms can be a capsule which can be of the ordinary hard- or soft-shelled gelatin type containing, for example, surfactants, lubricants, and inert fillers such as lactose, sucrose, calcium phosphate, and cornstarch.

In another embodiment, the pyrrolizidine compounds of the invention are tableted with conventional tablet bases such as lactose, sucrose, and cornstarch in combination with binders such as acacia, cornstarch, or gelatin, disintegrating agents intended to assist the break-up and dissolution of the tablet following administration such as potato starch, alginic acid, corn starch, and guar gum, lubricants intended to improve the flow of tablet granulations and to prevent the adhesion of tablet material to the surfaces of the tablet dies and punches, for example, talc, stearic acid, or magnesium, calcium, or zinc stearate, dyes, colouring agents, and flavouring agents intended to enhance the aesthetic qualities of the tablets and make them more acceptable to the patient.

Suitable excipients for use in oral liquid dosage forms include diluents such as water and alcohols, for example, ethanol, benzyl alcohol, and the polyethylene alcohols, either with or without the addition of a pharmaceutically acceptably surfactant, suspending agent or emulsifying agent.

The alkaloids of the invention may also be administered parenterally, that is, subcutaneously, intravenously, intramuscularly, or interperitoneally. In such embodiments, the alkaloid is provided as injectable doses in a physiologically acceptable diluent together with a pharmaceutical carrier (which can be a sterile liquid or mixture of liquids). Suitable liquids include water, saline, aqueous dextrose and related sugar solutions, an alcohol (such as ethanol, isopropanol, or hexadecyl alcohol), glycols (such as propylene glycol or polyethylene glycol), glycerol ketals (such as 2,2-dimethyl-1,3-dioxolane-4-methanol), ethers (such as poly(ethylene-glycol) 400), an oil, a fatty acid, a fatty acid ester or glyceride, or an acetylated fatty acid glyceride with or without the addition of a pharmaceutically acceptable surfactant (such as a soap or a detergent), suspending agent (such as pectin, carhomers, methylcellulose, hydroxypropylmethylcellulose, or carboxymethylcellulose), or emulsifying agent and other pharmaceutically adjuvants. Suitable oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamines acetates; anionic detergents, for example, alkyl, aryl, and olefin sulphonates, alkyl, olefin, ether, and monoglyceride sulphates, and sulphosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention will typically contain from about 0.5 to about 25% by weight of the alkaloid of the invention in solution. Preservatives and buffers may also be used. In order to minimize or eliminate irritation at the site of injection, such compositions may contain a non-ionic surfactant having a hydrophile-lipophile balance (HLB) of from about 12 to about 17. The quantity of surfactant in such formulations ranges from about 5 to about 15% by weight. The surfactant can be a single component having the above HLB or can be a mixture of two or more components having the desired HLB. Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The alkaloid of the invention may also be administered topically, and when done so the carrier may suitably comprise a solution, ointment or gel base. The base, for example, may comprise one or more of the following: petrolatum, lanolin, polyethylene glycols, bee wax, mineral oil, diluents such as water and alcohol, and emulsifiers and stabilizers. Topical formulations may contain a concentration of the compound from about 0.1 to about 10% w/v (weight per unit volume).

When used adjunctively, the alkaloid of the invention may be formulated for use with one or more other drug(s). In particular, the alkaloids may be used in combination with lysosomal enzymes adjunctive to enzyme replacement therapy. Thus, adjunctive use may be reflected in a specific unit dosage designed to be compatible (or to synergize) with the other drug(s), or in formulations in which the alkaloid is admixed with one or more enzymes. Adjunctive uses may also be reflected in the composition of the pharmaceutical kits of the invention, in which the alkaloids of the invention is co-packaged (e.g. as part of an array of unit doses) with the enzymes. Adjunctive use may also be reflected in information and/or instructions relating to the co-administration of the alkaloid and/or enzyme.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

### Identification of Pharmacoperones for α-Mannosidase

Fresh solutions had been prepared a day or so earlier of 0.2M Mcllvane buffer at pH 4.5 and 5 mM PNP-α-D-mannopyranoside (Sigma, N2127) in pH 4.5 buffer. Also prepared was a dilution of Jack bean α-D-mannosidase enzyme (Sigma, M7257, 22 Units/mg, 6.2 mg/ml.) at 0.6 Units/ml in pH 4.5 buffer.

The incubation mixture consisted of 10 μl enzyme solution, 10 μl of 1 mg/ml aqueous inhibitor solution and 50 μl of 5 mM substrate made up in buffer at the optimum pH for the enzyme. The reactions were stopped by addition of 70 μl 0.4M glycine (pH 10.4) during the exponential phase of the reaction, which had been determined at the beginning using uninhibited assays in which water replaced inhibitor. Final absorbances were read at 405 nm using a Versamax microplate reader (Molecular Devices). Assays were carried out in triplicate, and the values given are means of the three replicates per assay. Results were expressed as a percentage of uninhibited assays in which water replaced inhibitor.

Several polyhydroxylated alkaloids (imino sugars) were found to increase the activity of the enzyme by between 49% and 124% at the top concentration used (˜0.8 mM). The stimulation was so great in some cases that the absorbance values were above the linear range and so the compounds were repeated at 0.08 mM and absorbance values were within range and still showed stimulation from 7% to 30% for the diluted samples.

An assay was set up in which one of the compounds showing strong stimulation was mixed with an equal concentration of swainsonine and compared with swainsonine alone and as compound 1 alone. The swainsonine plus the selected compound and the swainsonine alone both gave 100% inhibition whereas the compound alone gave 90% stimulation.

Stimulation of other glycosidase activities such as α-glucosidase, α-galactosidase and hexosaminidases was also noted by a range of other imino sugars without them being inhibitory to any glycosidase tested. Such compounds might therefore have utility in diseases where specific glycosidase activities are deficient, including lysosomal storage disorders (Pompe's disease, Sandhoff's and Fabry's for example).

Many imino sugars have been observed by the inventors to increase the apparent activity of specific purified glycosidases. In the example given here we found that Jack Bean α-mannosidase activity (using p-nitrophenyl-α-D-mannopyranoside as the substrate) was greatly increased by certain imino sugars with a mannose configuration. These compounds did not cause inhibition of the mannosidase and swainsonine, a known inhibitor of this mannosidase, caused total inhibition of the promoted activity.

This study indicates that the catalytic site is free for binding of swainsonine and so we presume that the increased activity of the mannosidase is due to binding to another site on the enzyme. Swainsonine does not cause promotion of the mannosidase at any concentration tested.

### Example 2

### Identification of Pharmacoperones for Beta-Glucocerebrosidase

I. beta-glucocerebrosidase Activity Assay

Human Caucasian promyelocytic leukaemia cells (HL60, ECACC No. 98070106) were cultured using a standard sub-culture routine and lysed. The lysates were used as a source for wild type (wt) beta-glucocerebrosidase and used in an assay to determine the enzyme activity and conduct inhibition studies.

### i) Cell Lysate Preparation

HL60 cells were cultured to confluency and washed twice with PBS. Cells were lysed by the addition of lysis buffer (citric phosphate buffer (pH5.2), 0.1% Triton X-100, 0.25% taucholate) at 10×10⁶ cells/ml and incubated at 25° C. for 5 min. Lysates were cleared by centrifugation (400 g, 25° C., 5 min) and protein concentration was determined by using QuantiPro BCA assay kit (Sigma-Aldrich). Lysates were stored in aliquots at −80° C.

ii) beta-glucocerebrosidase Activity Assay

4-Methlyumbelliferyl β-D-glucopyranoside (4MU-β-D-glc) (Sigma) was used as a substrate to measure beta-glucocerebrosidase activity in HL60 lysate. Enzyme assays were performed in 96-well microtitre plates. Thawed cell lysate and 0.5 mM 4MU-β-D-glc in lysis buffer (50 μl final reaction volume) were mixed and incubated at 37° C. The reaction was quenched with 150 μl 0.5M sodium carbonate. The activity was measured by determining the rate of product (4MU) released using a fluorometer (OPTIMA, BMG) using excitation 360 nm, emission 450 nm filters. For inhibition studies, iminosugars at various concentrations (1 nM-100 μM) were co-incubated in the reaction mix.

### II. Enzyme Enhancement Assay—Cell Based Screening for Chaperones

Lymphoblasts derived from Gaucher's patients can be used for the cell based screening assays. EBV transformed B-lymphocytes from Gaucher's patients such as cell lines homozygous for the N370S mutation (GM01873) and L444P mutation (GM08752) in beta-glucocerebrosidase, were obtained from Coriell Institute for Medical Research. Cells were cultured in RMPI 1640 (Sigma) supplemented with 15% FBS (PAA), 2 mM L-glutamine and penicillin-streptomycin (PAA) as described in the culturing protocol.

Cells were seeded (8×10⁴ cells/well) and dosed (0.3-100 μM) in white 96-well plates (NUNC) to a final volume of 300 μL, and incubated for 72 hr at 37° C. in a 5% CO₂ incubator. Cells (200 μL) were transferred to 96-well Multiscreen harvester plates (Millipore) and harvested under vacuum. Cells were washed twice with PBS and lysed (and the enzyme reaction started) by the addition of 100 μL lysis buffer containing 5 mM 4MU-β-D-glc. Cell debris was removed by filtering through and collecting the cleared lysates. Lysates were incubated at 37° C. for a total time of 2 hrs. The enzyme reaction was quenched by addition of 150 μL 0.5M sodium carbonate to 50 μl of reaction mix. Fluorescence was measured as described above. QuantiPro BCA assay kit (Sigma) was used to determine the protein concentration in the cell lysates. Cell viability was measured using CellTiter-Glo® luminescent cell viability assay (Promega) on the remaining 100 μL unlysed cells. All experiments were performed in triplicates. The fold beta-glucocerebrosidase enzyme activity was determined relative to the vehicle (water or 1% DMSO) control, and normalised against total protein amount per well.

III. Identification of Non-Active Site Chaperones of beta-glucocerebrosidases

Compounds that demonstrated a significant increase in cellular beta-glucocerebrosidase activity (protocol II) but showed no direct inhibition of beta-glucocerebrosidase enzyme activity (protocol I) were considered to be non-active site chaperones.

Compounds identified according to the methods describe above find utility in the treatment of Gaucher's disease.

### Example 3

### Identification of Pharmacoperones for alpha-galactosidase

### I. Alpha-galactosidase Activity Assay

Human Caucasian promyelocytic leukaemia cells (HL60, ECACC No. 98070106) were cultured using a standard sub-culture routine and lysed. The lysates were used as a source for wild type (wt) alpha-galactosidase and used in an assay to determine the enzyme activity and conduct inhibition studies.

### i) Cell Lysate Preparation

Cell lysates were prepared as described above (Gaucher's I.i)

### ii) Alpha-galactosidase Activity Assay

4-Methlyumbelliferyl alpha-galactopyranoside (4MU-α-D-gal) (Sigma) was used as a substrate to measure alpha-galactosidase activity in HL60 lysate. Enzyme assays were performed in 96-well microtitre plates. Thawed cell lysate and 0.5 mM 4MU-α-D-gal in citric phosphate buffer (pH 4.5) containing 0.1M N-acetylgalactosamine (50 μl final reaction volume) were mixed and incubated at 37° C. The reaction was quenched with 150 μl 0.5M sodium carbonate. The activity was measured by determining the rate of product (4MU) released using a fluorometer (OPTIMA, BMG) using excitation 360 nm, emission 450 nm filters. For inhibition studies, iminosugars at various concentrations (1 nM-100 μM) were co-incubated in the reaction mix.

### II. Enzyme Enhancement Assay—Cell Based Screening for Chaperones

Lymphoblasts derived from Fabry's patients can be used for the cell based screening assays. EBV transformed B-lymphocytes from Fabry's patient (GM04391) were obtained from Coriell Institute for Medical Research. Cells were cultured in RMPI 1640 (Sigma) supplemented with 15% FBS(PAA), 2 mM L-glutamine and penicillin-streptomycin (PAA) as described in the culturing protocol.

Cells were seeded (8×10⁴ cells/well) and dosed (0.3-100 μM) in white 96-well plates (NUNC) to a final volume of 300 μL, and incubated for 72 hr at 37° C. in a 5% CO₂ incubator. Cells (200 μL) were transferred to 96-well Multiscreen harvester plates (Millipore) and harvested under vacuum. Cells were washed twice with PBS and lysed (and the enzyme reaction started) by the addition of 100 μL 5 mM 4MU-α-D-gal in citric phosphate buffer (pH4.5) with 0.1% Triton X-100 and 0.1M N-acetylgalactosamine (Sigma). Cell debris was removed by filtering through and collecting the cleared lysates, and the lysate was incubated at 37° C. for 2 hrs The enzyme reaction was quenched by addition of 150 μL 0.5M sodium carbonate to 50 μl of reaction mix. Fluorescence was measured as described above. Cell viability was measured using CellTiter-Glo® luminescent cell viability assay (Promega) on the remaining 100 μL unlysed cells. All experiments were performed in triplicates. The fold alpha-galactosidase enzyme activity was determined relative to the vehicle (water or 1% DMSO) control.

III. Identification of Non-Active Site Chaperones of alpha-galactosidase

Compounds that demonstrated a significant increase in cellular alpha-galactosidase activity (protocol II) but showed no direct inhibition of alpha-galactosidase enzyme activity (protocol I) were considered to be non-active site chaperones.

Compounds identified according to the methods describe above find utility in the treatment of Fabry's disease.

### Example 4

### Identification of Pharmacoperones for alpha-glucosidase

### I. Alpha-glucosidase Activity Assay

Human Caucasian promyelocytic leukaemia cells (HL60, ECACC No. 98070106) were cultured using a standard sub-culture routine and lysed. The lysates were used as a source for wild type (wt) lysosomal alpha-glucosidase and used in an assay to determine the enzyme activity and conduct inhibition studies.

### i) Cell Lysate Preparation

Cell lysates were prepared as described above (Gaucher's I.i)

### ii) Alpha-glucosidase Activity Assay

4-methlyumbelliferyl alpha-glucopyranoside (4MU-α-D-glc) (Sigma) was used as a substrate to measure alpha-glucosidase activity in HL60 lysate. Enzyme assays were performed in 96-well microtitre plates. Thawed cell lysate and 0.5 mM 4MU-α-D-glc in citric phosphate buffer (pH 4.5) (50 μl final reaction volume) were mixed and incubated at 37° C. The reaction was quenched with 150 μl 0.5M sodium carbonate. The activity was measured by determining the rate of product (4MU) released using a fluorometer (OPTIMA, BMG) using excitation 360 nm, emission 450 nm filters. For inhibition studies, iminosugars at various concentrations (1 nM-100 μM) were co-incubated in the reaction mix.

Compounds that demonstrated an increase in cellular alpha-glucosidase activity (over 1.2 fold) (protocol II) but showed no direct inhibition of alpha-glucosidase enzyme activity (protocol I) were considered to be non-active site chaperones.

Compounds identified according to the methods describe above find utility in the treatment of Pompe's disease.

### II. Enzyme Enhancement Assay—Cell Based Screening for Chaperones

Lymphoblasts derived from Pompe's patients can be used for the cell based screening assays. EBV transformed B-lymphocytes from Pompe's patient such as (GM013963) and (GM06314) were obtained from Coriell Institute for Medical Research. Cells were cultured in RMPI 1640 (Sigma) supplemented with 15% FBS(PAA), 2 mM L-glutamine and penicillin-streptomycin (PAA) as described in the culturing protocol.

Cells were seeded (8×10⁴ cells/well) and dosed (0.3-100 μM) in white 96-well plates (NUNC) to a final volume of 300 μL, and incubated for 72 hr at 37° C. in a 5% CO₂ incubator. Cells (200 μL) were transferred to 96-well Multiscreen harvester plates (Millipore) and harvested under vacuum. Cells were washed twice with PBS and lysed (and the enzyme reaction started) by the addition of 100 μL 5 mM 4MU-α-D-glc in citric phosphate buffer (pH4.5) with 0.1% Triton X-100 (Sigma). Cell debris was removed by filtering through and collecting the cleared lysates, and the lysate was incubated at 37° C. for 2 hrs. The enzyme reaction was quenched by addition of 150 μL 0.5M sodium carbonate to 50 μl of reaction mix. Fluorescence was measured as described above. Cell viability was measured using CellTiter-Glo® luminescent cell viability assay (Promega) on the remaining 100 μL unlysed cells. All experiments were performed in triplicates. The fold alpha-glucosidase enzyme activity was determined relative to the vehicle (water or 1% DMSO) control.

III. Identification of Non-Active Site Chaperones of alpha-glucosidase

Compounds that demonstrated a significant increase in cellular alpha-glucosidase activity (protocol II) but showed no direct inhibition of alpha-glucosidase enzyme activity (protocol I) were considered to be non-active site chaperones.

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1-36. (canceled)

37. A method of treating or preventing a disease or disorder arising from abnormal protein folding in a mammalian cell, said method comprising administering to a subject in need thereof a polyhydroxylated alkaloid or imino sugar which is a pharmacoperone of a protein and which does not bind to a catalytic site of an enzyme in an amount effective to enhance normal folding of the protein.

38. The method of claim 37 wherein the disease or disorder arising from abnormal protein folding is a lysosomal storage disease, for example a lysosomal storage disease selected from the group consisting of: (a) Pompe's disease; (b) Gaucher's disease; (c) Fabry's disease; (d) GMI-gangliosidosis; (e) Tay-Sachs' disease; (f) Sandhoff's disease; (g) Niemann-Pick's disease; (h) Krabbe's disease; (i) Farber's disease; (j) Metachromatic leukodystrophy; (k) Hurler-Scheie's disease; (l) Hunter's disease; (m) Sanfilippo's disease A, B, C or D; (n) Morquio's disease A or B; (o) Maroteaux-Lamy's disease; (p) Sly's disease; (q) alpha-Mannosidosis; (r) beta-Mannosidosis; (s) Fucosidosis; (t) Sialidosis; and (u) Schindler-Kanzaki's disease.

39. The method of claim 38 wherein the polyhydroxylated alkaloid or imino sugar is a pharmacoperone of an enzyme selected from the group consisting of: (a) Acid alpha-glucosidase; (b) Acid beta-glucosidase; (c) glucocerebrosidase; (d) alpha-Galactosidase A; (e) Acid beta-galactosidase; (f) beta-Hexosaminidase A; (g) beta-Hexosaminidase B; (h) Acid sphingomyelinase; (i) Galactocerebrosidase; (j) Acid ceramidase; (k) Arylsulfatase A; (l) alpha-L-Iduronidase; (m) Iduronate-2-sulfatase; (n) Heparan N-sulfatase; (o) alpha-N-Acetylglucosaminidase; (p) Acetyl-CoA: alpha-glucosaminide N-acetyltransferase; (q) N-Acetylglucosamine-6-sulfate sulfatase; (r) N-Acetylgalactosamine-6-sulfate sulfatase; (s) Acid beta-galactosidase; (t) Arylsulfatase B; (u) beta-Glucuronidase; (v) Acid alpha-mannosidase; (w) Acid beta-mannosidase; (x) Acid alpha-L-fucosidase; (y) Sialidase; and (z) alpha-N-acetylgalactosaminidase.

40. The method of claim 37 wherein the polyhydroxylated alkaloid or imino sugar is a bicyclic polyhydroxylated alkaloid.

41. The method of claim 40 wherein the alkaloid or imino sugar is selected from a structural class selected from the group consisting of:
(a) a piperidine alkaloid;
(b) a pyrroline alkaloid;
(c) a pyrrolidine alkaloid;
(d) a pyrrolizidine alkaloid;
(e) an indolizidine alkaloid;
(f) a quinolizidine alkaloid;
(g) a nortropane alkaloid;
(h) mixtures of any two or more of (a) to (g).

42. The method of claim 37 wherein the alkaloid is an imino sugar or imino sugar acid.

43. The method of claim 42 wherein the alkaloid or imino sugar is selected from the group consisting of:
(a) a glycoside derivative;
(b) a branched alkyl derivative;
(c) a derivative in which one or more of the hydroxyl group(s) are masked or protected;
(d) a glucose analogue;
(e) a galactose analogue;
(f) a mannose analogue.

44. A polyhydroxylated alkaloid or imino sugar which is a pharmacoperone of an enzyme and which does not bind to a catalytic site of said enzyme.

45. The pharmacoperone of claim 44 which is not a competitive inhibitor of said enzyme.

46. The pharmacoperone of claim 45 which is an activator of said enzyme.

47. The pharmacoperone of claim 45 which is a non-competitive inhibitor of said enzyme.

48. The pharmacoperone of claim 44 which binds to an allosteric site of said enzyme.

49. The pharmacoperone of claim 44 which does not bind to said enzyme but binds to a chaperone or co-chaperone of said enzyme.

50. A pharmaceutical composition comprising the pharmacoperone of claim 44 together with a pharmaceutical excipient.

51. The pharmacoperone of claim 44 wherein the polyhydroxylated alkaloid or imino sugar is a bicyclic polyhydroxylated alkaloid.

52. The pharmacoperone of claim 44 wherein the alkaloid or imino sugar is selected from a structural class selected from the group consisting of:
(a) a piperidine alkaloid;
(b) a pyrroline alkaloid;
(c) a pyrrolidine alkaloid;
(d) a pyrrolizidine alkaloid;
(e) an indolizidine alkaloid;
(f) a quinolizidine alkaloid;
(g) a nortropane alkaloid;
(h) mixtures of any two or more of (a) to (g).

53. The pharmacoperone of claim 44 wherein the alkaloid is an imino sugar or imino sugar acid.

54. The pharmacoperone of claim 53 wherein the alkaloid or imino sugar is selected from the group consisting of:
(a) a glycoside derivative;
(b) a branched alkyl derivative;
(c) a derivative in which one or more of the hydroxyl group(s) are masked or protected;
(d) a glucose analogue;
(e) a galactose analogue;
(f) a mannose analogue.
